(19) United States
(12) Patent Application Publication
(10) Pub. No.: US 2023/0329527 A1
(43) Pub. Date: Oct. 19, 2023

(54) MEDICAL DEVICES AND RELATED SYSTEMS AND METHODS

(71) Applicants: Boston Scientific Medical Device Limited, Galway (IE); Boston Scientific Scimed, Inc., Maple Grove, MN (US)

(72) Inventors: Aditya DHANOTIYA, Indore (IN); Nabarun BHOWMICK, Kolkata (IN); Shrikant Vasant RAUT, Mumbai (IN); James WELDON, Newton, MA (US)

(73) Assignees: Boston Scientific Medical Device Limited, Galway (IE); Boston Scientific Scimed, Inc., Maple Grove, MN (US)

(21) Appl. No.: 18/302,131

(22) Filed: Apr. 18, 2023

Related U.S. Application Data
(60) Provisional application No. 63/363,200, filed on Apr. 19, 2022.

(51) Int. Cl. A61B 1/00

(52) U.S. Cl. CPC A61B 1/00066

(57) ABSTRACT

A handle assembly for a medical device is disclosed. The handle assembly may comprise a handle body including a first central longitudinal axis, a first connector portion coupled to a distal end of the handle body, and a shaft extending distally from the first connector portion, wherein a second central longitudinal axis of the shaft is offset from the first central longitudinal axis of the handle body.

### CROSS-REFERENCE TO RELATED APPLICATIONS

This application claims the benefit of priority of U.S. Provisional Patent Application No. 63/363,200, filed Apr. 19, 2022, the entirety of which is incorporated herein by reference.

### TECHNICAL FIELD

Various aspects of this disclosure relate generally to handles of medical devices. More specifically, embodiments of this disclosure relate to handles of an endoscope or other medical device, including an endoscope or other medical device having offset handle and shaft axes, among other aspects.

### BACKGROUND

During endoscopic procedures, a medical professional operating an endoscope often wraps his/her entire palm around a grip or handle portion of a medical device, such as an endoscope. Various actuators on the handle of the endoscope require the medical professional to contort his/her hands frequently and for prolonged periods of time during a procedure, which can cause strain, or even an injury. In some cases, actuation of different scope controls, like knobs or an elevator, may result in excessive movements of the medical professional's thumb or other fingers, which may result in strain in the medical professional's hand. Endoscope operators can experience wrist and hand discomfort resulting from holding and manipulating the endoscope's handle. In some cases, for example, medical professionals may experience symptoms similar to those of Carpal Tunnel Syndrome or tendonitis. When a medical professional experiences fatigue or other pain in the fingers, hand, or wrist, the medical professional may shift from a primary grip position to a secondary grip position that may be a less powerful grip than the primary grip position, such as shifting from a four finger grip to a three finger grip. Repeatedly bending and maneuvering of the wrist to adjust positioning of the endoscope can increase fatigue or other pain.

When a medical professional repeatedly readjusts his or her hand grip in between procedure tasks, the procedure may be prolonged, and procedural tasks may be more difficult. Depending on the size of a medical professional's hand, actuators may be positioned in non-optimized positions and increase the number of readjustments of the professional's hand during a procedure.

### SUMMARY

Aspects of the disclosure relate to, among other things, systems, devices, and methods to help reduce strain on a user's hand during operating of a handle assembly. The systems, devices, and methods of this disclosure may facilitate a user's hand grip on an endoscope or other medical device. Endoscopes and other medical devices with a handle that may rotate relative to a proximal shaft portion may help address user hand fatigue or strain, may help reduce the need for hand grip readjustments, and may help address other issues. Each of the aspects disclosed herein may include one or more of the features described in connection with any of the other disclosed aspects.

According to one aspect, a handle assembly for a medical device is disclosed. The handle assembly may comprise a handle body including a first central longitudinal axis, a first connector portion coupled to a distal end of the handle body, and a shaft extending distally from the first connector portion, wherein a second central longitudinal axis of the shaft is offset from the first central longitudinal axis of the handle body.

In other aspects, the handle assembly may include one or more of the following features. A second connector portion may be coupled to (i) a proximal end of the shaft and (ii) the first connector portion, and the second connector portion may be spaced from the first central longitudinal axis of the handle body. The shaft may further comprises a distal tip and a camera positioned at the distal tip. A first interior cavity of the handle body may be in communication with to a second interior cavity of the first connector portion. Internal components may extend longitudinally through the first interior cavity and the second interior cavity. The shaft may include a strain relief portion at a proximal portion of the shaft. The first connector portion may include a working channel port. At least a portion of the first connector portion may extend from a first side of the handle body to a second side of the handle body, and may protrude radially outward from the second side of the handle body. The first connector portion may include a proximal-facing planar surface. The handle body may include (a) a suction actuator and (b) an air and water actuator. The handle body may include a first control knob, a second control knob, a locking lever, and a locking knob. The central longitudinal axis of the handle body may be substantially parallel to the central longitudinal axis of the shaft. The second connector portion may extend into (i) the first connector portion and (ii) the shaft. The connector may be rectangular. The central longitudinal axis of the handle body may be approximately 68 mm from the central longitudinal axis of the shaft.

In other aspects, a handle assembly for a medical device may comprise a handle body including a central longitudinal axis, a first connector portion coupled to a distal end of the handle body, a second connector portion coupled to a distal portion of the first connector portion, positioned entirely distal to the handle body, and spaced from the handle body; and a shaft extending distally from the second connector portion, wherein a central longitudinal axis of the shaft is spaced from the central longitudinal axis of the handle body.

In other aspects, the handle assembly may include one or more of the following features. The handle assembly may be configured to be held by a user such that a wrist of the user is aligned with the central longitudinal axis of the shaft. The central longitudinal axis of the handle body may be substantially parallel to the central longitudinal axis of the shaft. The handle body may include a first control knob, a second control knob, a locking lever, and a locking knob.

In other aspects, a method of operating an endoscope that includes a handle is disclosed. The method may comprise moving a handle body circumferentially about a central longitudinal axis of a shaft of the endoscope. The user's wrist aligns with the central longitudinal axis of the shaft; and the handle body is spaced from the central longitudinal axis of the shaft.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIGS. 1A and 1B are perspective views of an exemplary endoscope, according to aspects of this disclosure.

FIGS. 2A and 2B are perspective views of a user's hand holding an endoscope handle (such as a handle of the endoscope of FIGS. 1A and 1B), according to aspects of this disclosure.

FIG. 3 is a side view of a portion of an endoscope including a handle assembly, according to aspects of this disclosure.

FIG. 4 is a side view of a portion of an endoscope including a handle assembly, having certain transparent portions to reveal inner structures, according to aspects of this disclosure.

FIG. 5 is a side, partial cross-sectional view of a portion of the handle assembly of FIGS. 3 and 4, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. Throughout the figures included in this application, arrows labeled “P” and “D” are used to show the proximal and distal directions in the figure. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

Embodiments of this disclosure seek to improve a user's ability to grip, manipulate, and/or otherwise apply force to a handle, and actuators of the handle, of a medical device (such as an endoscope), during a medical procedure. As non-limiting exemplary benefits, aspects of this disclosure may reduce the need to reposition the user's hand during a procedure and reduce strain to the user's hand from excessive movement of fingers, among other aspects.

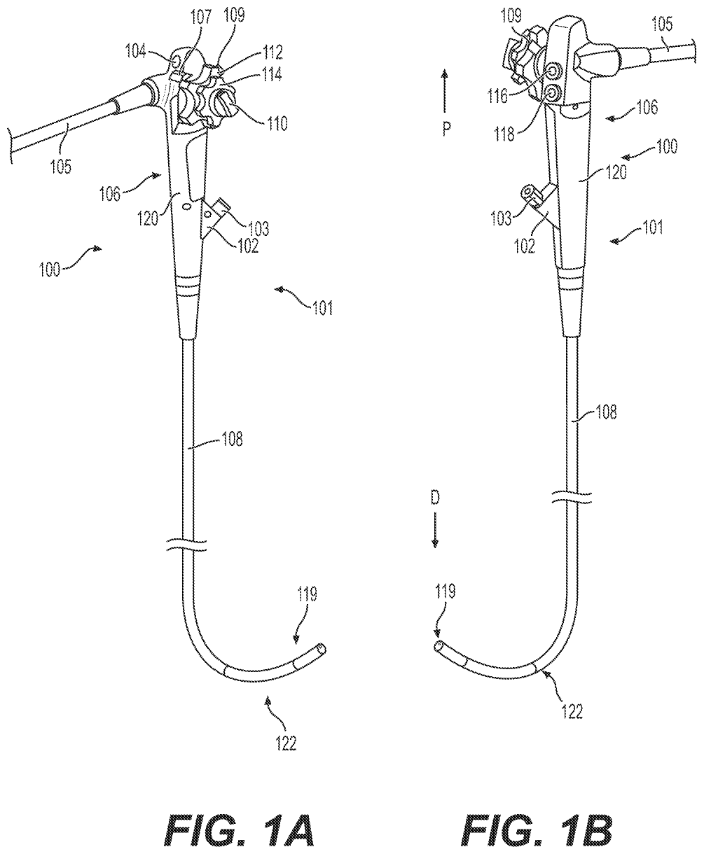

FIGS. 1A and 1B show perspective views of an exemplary endoscope system 100. Endoscope system 100 may include an endoscope 101. Although the term endoscope may be used herein, it will be appreciated that other devices, including, but not limited to, duodenoscopes, colonoscopes, ureteroscopes, bronchoscopes, laparoscopes, sheaths, catheters, or any other suitable delivery device or other type of medical device may be used in connection with the devices of this disclosure, and the handle assembly discussed below may be incorporated into any of these or other medical devices.

Endoscope 101 may include a handle assembly 106 and a flexible tubular shaft 108. The handle assembly 106 may include a biopsy port 102, a biopsy cap 103, an image capture button 104, an elevator actuator 107, a locking lever 109, a locking knob 110, a first control knob 112, a second control knob 114, a suction button 116, an air/water button 118, a handle body 120, and an umbilicus 105. All of the actuators, elevators, knobs, buttons, levers, ports, or caps of endoscope system 100, such as those enumerated above, may serve any purpose and are not limited by any particular use that may be implied by the respective naming of each component used herein. The umbilicus 105 may extend from handle body 120 to auxiliary devices, such as a control unit, water/fluid supply, and/or vacuum source. Umbilicus 105 may transmit signals between endoscope 101 and the control unit, in order to control lighting and imaging components of endoscope 101 and/or receive image data from endoscope 101. Umbilicus 105 also can provide fluid for irrigation from the water/fluid supply and/or suction to a distal tip 119 of shaft 108. Buttons 116 and 118 control valves for suction and fluid supply (e.g., air and water), respectively. Shaft 108 may terminate at distal tip 119. Shaft 108 may include an articulation section 122 for deflecting distal tip 119 in up, down, left, and/or right directions. Knobs 112 and 114 may be used for controlling such deflection. Locking lever 109 and locking knob 110 may lock knobs 112 and 114, respectively, in desired positions. Handle body 120 may be tapered and may narrow as the handle extends distally such that the profile of handle body 120 is smaller at its distal end than at its proximal end.

Distal tip 119 may include an imaging device (e.g., a camera) and a lighting source (e.g., an LED or an optical fiber). Distal tip 119 may be side-facing. That is, the imaging device and the lighting source may face radially outward, perpendicularly, approximately perpendicularly, or otherwise transverse to a longitudinal axis of shaft 108 and distal tip 119. In alternatives, distal tip 119 may be forward-facing. That is, the imaging device and the lighting device may face approximately along a longitudinal axis of distal tip 119 and shaft 108.

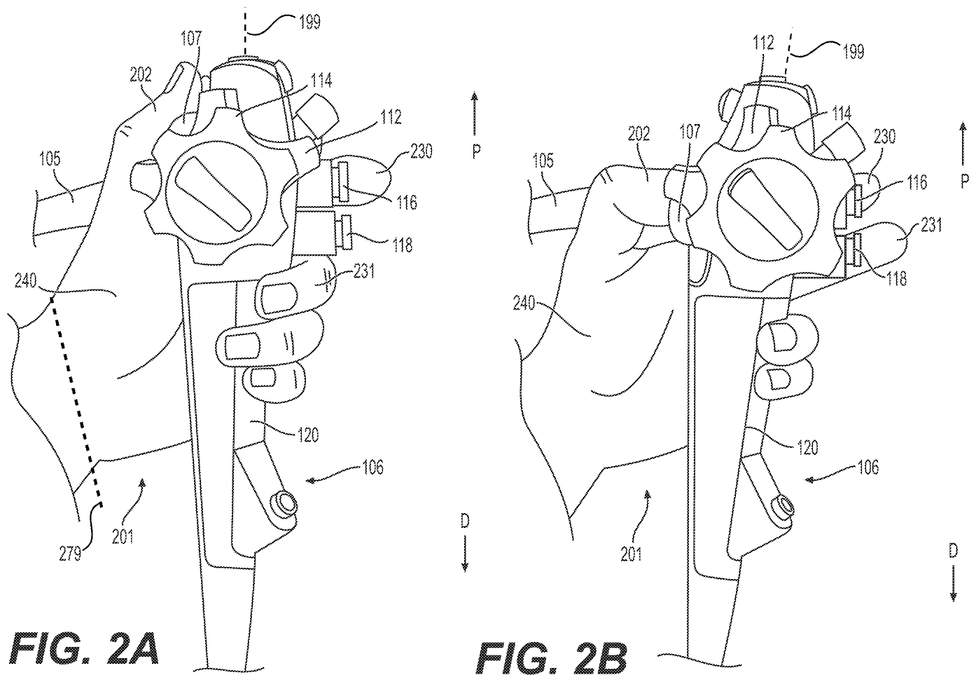

In operating endoscope system 100, a user may use his/her left hand to hold the handle assembly 106 (shown in FIG. 2A) while the right hand is used to hold accessory devices and/or operate one or more of the actuators of the handle assembly 106, such as the first and second control knobs 112, 114 and the locking lever 109 and locking knob 110. The user may grasp the handle assembly 106 by wrapping the user's hand around the handle body 120. When grasping handle body 120, the user may use the left thumb to operate the first and second control knobs 112, 114 and the elevator actuator 107 (through rotation about their respective axes), and may use a left-hand finger to operate the image capture button 104, the suction button 116, and/or the air/water button 118 (each by pressing).

FIGS. 2A and 2B show an exemplary user's left hand 201 grasping handle assembly 106, which may have a central longitudinal axis 199 extending longitudinally through handle assembly 106. The user may position the thumb 202 of the left hand 201 (the grasping hand) over the elevator actuator 107 and move the elevator actuator 107 along a circular path from a first position (shown in FIG. 2A) to a second position (shown in FIG. 2B) by moving the thumb 202. FIG. 2A illustrates an axis 279 aligned with the wrist radial-ulnar joint axis, or the axis that hand 201 rotates about when the user moves their wrist. For example, a user may rotate shaft 108 by bending their wrist to rotate handle assembly 106. The handle assembly discussed in this disclosure may facilitate grasping the handle, may reduce the strain on a user's hand when repositioning the medical device, and may facilitate rotation of the medical device using the user's hand and wrist.

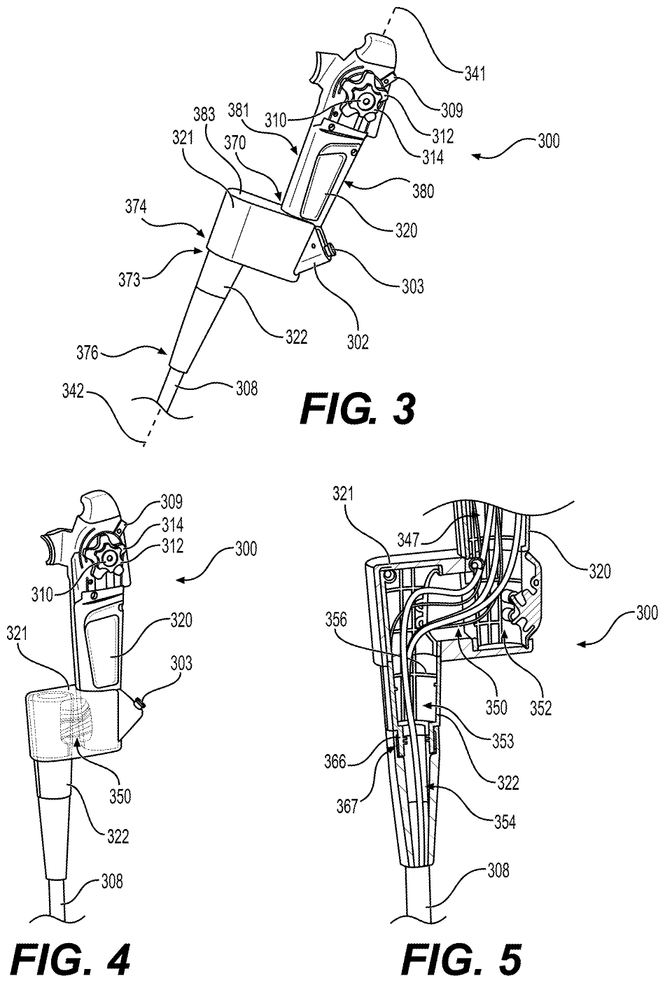

FIG. 3 shows a perspective view of an alternative handle assembly 300 including a locking lever 309, a locking knob 310, a first control knob 312, a second control knob 314, a handle body 320, and a shaft 308. Handle assembly 300 may include any of the features of handle assemblies 106. A distal end portion 370 of handle body 320 may be coupled to a first connector portion 321. First connector portion 321 may include working channel port 302 with working channel opening 303. A proximal end 373 of a second connector portion 322 may be coupled to a distal portion 374 of first connector portion 321, and shaft 308 may include a strain relief portion 376. Handle body 320 may have a central longitudinal axis 341, and shaft 308 may have a central longitudinal axis 342 offset from axis 341 of handle body 320. In some examples, axis 341 may be substantially parallel to axis 342. Axis 341 may be spaced from axis 342 approximately 68 mm.

The offset of handle body 320 from shaft 308 (i.e., the offset of axis 341 from axis 342) may facilitate a user applying torque to shaft 308 by moving handle body 320. In some examples, when a user grips handle body 320, the user's radial-ulnar joint axis 279 may be substantially aligned with (i.e., substantially coaxial with) central longitudinal axis 342 of shaft 308, which may facilitate rotating shaft 308 about axis 342 when a user bends his or her wrist. As shaft 308 rotates about axis 342, handle body 320 may move circumferentially about axis 342. In other words, first connector portion 321 and handle body 320 may pivot about axis 342.

At least a portion of first connector portion 321 may extend across the distal end portion 370 of handle body 320 from a first side 380 of handle body 320 to a second side 381 of handle body 320, and protrude radially outward, relative to axis 341, from second side 381. For example, a distal end of A proximal end 383 of first connector portion 321 may be substantially planar and may abut the distal end portion 370 of handle body 320. Proximal end 282 may include a proximally-facing planar surface. In some examples, first connector portion 321 may include a first half portion and a second half portion that are fastened together to form first connector portion 321.

Working channel port 302 may be adjacent to the first side 380 of handle body 320, and may be spaced from second connector portion 322. As will be discussed further in relation to FIG. 5, portion of second connector portion 322 may be received within first connector portion 321. Shaft 308 may include a strain relief portion 376 coupled to a distal end of second connector portion 322.

FIG. 4 illustrates a perspective view of handle assembly 300 with first connector portion 321 shown as transparent to expose internal components 350 of handle assembly 300. Internal components 350, such as air/water and/or suction tubes, electrical wiring, a working channel tube, articulation wires configured to move a distal portion of shaft 308, and other components, may extend through first connector portion 321 and second connector portion 322 into shaft 308.

FIG. 5 shows a partial cross-sectional view of a portion of handle assembly 300 including handle body 320, first connector portion 321, second connector portion 322, and shaft 308. As shown in FIG. 5, second connector portion 322 extends proximally into first connector portion 321. An interior cavity 347 of handle body 320 may open into an interior cavity 352 of first connector portion 321, and the interior cavity 352 of first connector portion 321 may open into an interior cavity 353 of second connector portion 322, such that each of interior cavities 47, 352, 353 are fluidically connected. The opening of first connector portion 321 in communication with interior cavity 347 is substantially coaxial with axis 341, while the opening of first connector portion 321 in communication with interior cavity 353 is substantially coaxial with axis 342, for example. The proximal wall of the first connector portion 321 extends from a far side of second connector portion 322 (relative to 320) to second side 381. The distal wall of the first connector portion 321 extends from a close side of second connector portion 322 (relative to 320) to first side 380. Interior cavity 353 may be connected to an interior lumen 354 of shaft 308. In some examples, a distal portion 366 of second connector portion 322 may be received within a proximal end portion 367 of lumen 354. Components 350 of handle assembly 300 may pass through first connector portion 321 and second connector portion 322 into shaft 308.

In operation, a user may rotate the first control knob 312 to move a distal tip of shaft 308 (or an articulation section of shaft 308) to the right or left, may rotate the second control knob 314 to move the distal tip of shaft 308 upward or downward, may actuate locking lever 309 to brake or prevent movement of the distal tip in the right and left directions, and may actuate locking knob 314 to brake or prevent movement of the distal tip in the up and down directions. A user may rotate shaft 308 by pivoting handle body 320 about central longitudinal axis 342 of shaft 308. For example, the user may pivot their wrist while holding handle body 320, the radial-ulnar joint axis 279 of the user's gripping hand may be substantially aligned with central longitudinal axis 342 of shaft 308, and shaft 308 my rotate about axis 342. Since handle body 320 is offset from shaft 308, a user may move handle body 320 circumferentially about axis 342 to rotate shaft about axis 342 and first connecting portion 321 may act as a lever and increase the amount of torque applied to shaft 308. Since the amount of torque required to rotate shaft 308 via handle body 320 is reduced compared to conventional handle assemblies (such as handle assembly 106), handle assembly 300 may reduce the amount of strain on a user's hand and wrist during operation because the amount of torque required to rotate shaft 308 is decreased.

Handle assembly 300 may reduce hand strain caused by excessive movement of a user's wrist when the user operates endoscope 101, and may reduce the chance of the user losing his or her grip. Also, the handle assembly 300 may help prevent repeated repositioning of a user's hand on a medical device handle due to fatigue, strain, or other difficulty. Each of the aforementioned handle assemblies 106, 300, whether used in conjunction with an endoscope system or any other medical device, may be used to enhance and/or facilitate a user's grip on a handle and/or a user's ability to manipulate the medical device. Any portion of the handle assemblies or actuators discussed herein may be incorporated into a handle of an endoscope or other medical device to improve a user's operation of the device.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A handle assembly for a medical device comprising:
a handle body including a first central longitudinal axis,
a first connector portion coupled to a distal end of the handle body, and
a shaft extending distally from the first connector portion, wherein a second central longitudinal axis of the shaft is offset from the first central longitudinal axis of the handle body.

2. The handle assembly of claim 1, further comprising a second connector portion coupled to (i) a proximal end of the shaft and (ii) the first connector portion, wherein the second connector portion is spaced from the first central longitudinal axis of the handle body.

3. The handle assembly of claim 2, wherein the shaft further comprises:
a distal tip; and
a camera positioned at the distal tip.

4. The handle assembly of claim 1, wherein a first interior cavity of the handle body is in communication with to a second interior cavity of the first connector portion.

5. The handle assembly of claim 4, further comprising internal components extending longitudinally through the first interior cavity and the second interior cavity.

6. The handle assembly of claim 1, wherein the shaft includes a strain relief portion at a proximal portion of the shaft.

7. The handle assembly of claim 1, wherein the first connector portion includes a working channel port.

8. The handle assembly of claim 1, wherein at least a portion of the first connector portion extends from a first side of the handle body to a second side of the handle body, and protrudes radially outward from the second side of the handle body.

9. The handle assembly of claim 1, wherein the first connector portion includes a proximal-facing planar surface.

10. The handle assembly of claim 1, wherein the handle body includes (a) a suction actuator and (b) an air and water actuator.

11. The handle assembly of claim 10, wherein the handle body includes a first control knob, a second control knob, a locking lever, and a locking knob.

12. The handle assembly of claim 1, wherein the central longitudinal axis of the handle body is substantially parallel to the central longitudinal axis of the shaft.

13. The handle assembly of claim 2, wherein the second connector portion extends into (i) the first connector portion and (ii) the shaft.

14. The handle assembly of claim 1, wherein the connector is rectangular.

15. The handle assembly of claim 1, wherein the central longitudinal axis of the handle body is approximately 68 mm from the central longitudinal axis of the shaft.

16. A handle assembly for a medical device comprising:
a handle body including a central longitudinal axis,
a first connector portion coupled to a distal end of the handle body,
a second connector portion coupled to a distal portion of the first connector portion, positioned entirely distal to the handle body, and spaced from the handle body; and
a shaft extending distally from the second connector portion, wherein a central longitudinal axis of the shaft is spaced from the central longitudinal axis of the handle body.

17. The handle assembly of claim 16, wherein the handle assembly is configured to be held by a user such that a wrist of the user is aligned with the central longitudinal axis of the shaft.

18. The handle assembly of claim 16, wherein the central longitudinal axis of the handle body is substantially parallel to the central longitudinal axis of the shaft.

19. The handle assembly of claim 16, wherein the handle body includes a first control knob, a second control knob, a locking lever, and a locking knob.

20. A method of operating an endoscope that includes a handle, the method comprising:
moving a handle body circumferentially about a central longitudinal axis of a shaft of the endoscope, wherein the user's wrist aligns with the central longitudinal axis of the shaft;
wherein the handle body is spaced from the central longitudinal axis of the shaft.
